(19) United States
(12) Patent Application Publication
(10) Pub. No.: US 2016/0151021 A1
(43) Pub. Date: Jun. 2, 2016

(54) SYSTEM AND METHOD FOR DETECTING DRIVER'S SUDDEN HEART ATTACK

(71) Applicant: AUTOMOTIVE RESEARCH & TEST CENTER, CHANGHUA COUNTY 50544 (TW)

(72) Inventors: YEN-CHENG FENG, CHANGHUA COUNTY 50544 (TW); MING-KUAN KO, CHANGHUA COUNTY 50544 (TW)

(21) Appl. No.: 14/584,024

(22) Filed: Dec. 29, 2014

(30) Foreign Application Priority Data
Nov. 28, 2014 (TW) 103141510

(51) Int. Cl. A61B 5/00; A61B 5/0205

(52) U.S. Cl. CPC A61B 5/6893; A61B 5/0205; A61B 5/0002; A61B 5/7267; A61B 5/7275; A61B 5/746; A61B 5/7282; A61B 5/021

(57) ABSTRACT

A system and method for detecting a driver's sudden heart attack applies to detecting whether the driver of a vehicle is suffering a sudden heart attack. The method comprises steps: persistently capturing vital-sign signals of respiration rate, heart rhythm and blood pressure of the driver and transmitting the vital-sign signals to a monitoring system; a processor of the monitoring system using an artificial neural network to train the vital-sign signals and establish personalized models specific to the driver and respectively having threshold values; the monitoring system examining whether any one of the vital-sign signals exceeds its threshold value; if at least one type of the vital-sign signals exceeds the threshold value thereof, determining a risk level of the driver according to the quantity of the types of the vital-sign signals exceeding the threshold values thereof, and emitting an alert if necessary.

This application claims priority for Taiwan patent application no. 103141510 filed at Nov. 28, 2014, the content of which is incorporated by reference in its entirely.

### BACKGROUND OF THE INVENTION

1. Field of the Invention

The present invention relates to a driver's status detection technology, particularly to a system and method for detecting a driver's sudden heart attack.

2. Description of the Related Art

In addition to traffic rule violation (such as speeding and wrong way driving), traffic accidents are also attributed to attention deficit and sudden diseases. Attention deficit may be due to fatigue, talking or phoning while driving, which can all be avoided by drivers if they are willing to do. Contrarily, sudden diseases, such as heart attack, coma and sudden death, are unpredictable and unpreventable. No matter whether a suddenly-disabled driver stops the vehicle on the road abruptly or does not release the accelerator but leaves the vehicle running on the road, these are all very dangerous behaviors. A driver in coma may even push hard the accelerator unconsciously and cause a serious overtaking collision.

Since sudden diseases are unpredictable, it is very important to take appropriate measures in the moment a sudden disease occurs. It is first of all to detect whether the driver is suffering a sudden disease, especially a sudden heart attack, because a sudden heart attack is most dangerous to the driver's life and the traffic safety. Suppose a system can detect a driver's sudden heart attack, turn off the engine, and flash the lights, or even park the vehicle on the side of the road and transmit emergency information to the police station or the hospital, the system not only can prevent from a traffic accident but also can possibly save the driver's life.

Accordingly, the present invention proposes a system and method for detecting a driver's sudden heart attack. The principles and embodiments of the present invention will be described in detail below.

### SUMMARY OF THE INVENTION

The primary objective of the present invention is to provide a system for detecting a driver's sudden heart attack, which uses a plurality of sensors to detect the vital signs of the driver, including heart rhythm, blood pressure and respiration rate, to acquire the physiological status of the driver and determine whether the driver is suffering a sudden heart attack.

Another objective of the present invention is to provide a method for detecting a driver's sudden heart attack, which trains an artificial neural network to establish customized models of respiration rate, blood pressure and heart rhythm, which are specific to an individual driver, whereby to increase the accuracy of predicting a sudden heart attack and provide the physicians with the vital sign information persistently captured by the system when the driver goes to a hospital.

A further objective of the present invention is to provide a method for detecting a driver's sudden heart attack, which examines whether the other two vital-sign signals are abnormal while at least one vital-sign signal has exceeded a threshold value to determine whether the driver is suffering a sudden disease and must go to a hospital immediately.

In order to achieve the abovementioned objectives, the present invention proposes a method for detecting a driver's sudden heart attack, which comprises steps: persistently capturing a plurality of vital-sign signals of the driver, including a respiration rate signal, a heart rhythm signal and a blood pressure signal, and transmitting the vital-sign signals to a monitoring system; the processor of the monitoring system using the vital-sign signals to train an artificial neural network to establish a plurality of personalized models, including a respiration rate model, a heart rhythm model and a blood pressure model, which are specific to the driver and respectively have threshold values; the monitoring system examining whether any vital-sign signal exceeds the threshold value thereof; if none vital-sign signal exceeds its threshold value, continuing capturing the vital-sign signals; if at least one vital-sign signal exceeds the threshold value thereof, the monitoring system determining the risk of the driver according to the number of the types of the vital-sign signals exceeding the threshold values thereof and emitting an alert if necessary.

The present invention also proposes a system for detecting a driver's sudden heart attack, which comprises a plurality of sensors persistently capturing a plurality of vital-sign signals of the driver, including a respiration rate signal, a heart rhythm signal and a blood pressure signal; and a monitoring system including a processor and a memory. The processor uses the vital-sign signals to train an artificial neural network to establish a plurality of personalized models, including a respiration rate model, a heart rhythm model and a blood pressure model, and stores the models in the memory. The processor respectively sets threshold values of the respiration rate model, the heart rhythm model and the blood pressure model. The processor examines whether any vital-sign signal exceeds the threshold value thereof. If at least one vital-sign signal exceeds the threshold value thereof, the monitoring system determines the risk of the driver according to the number of the types of the vital-sign signals exceeding the threshold values thereof and emits an alert if necessary.

Below, embodiments are described in detail to make easily understood the objectives, technical contents, characteristics and accomplishments of the present invention.

### BRIEF DESCRIPTION OF THE DRAWINGS

FIG. 1 is a block diagram schematically showing a system for detecting a drive's sudden heart attack according to one embodiment of the present invention; and

FIG. 2 is a flowchart of a method for detecting a drive's sudden heart attack according to one embodiment of the present invention.

### DETAILED DESCRIPTION OF THE INVENTION

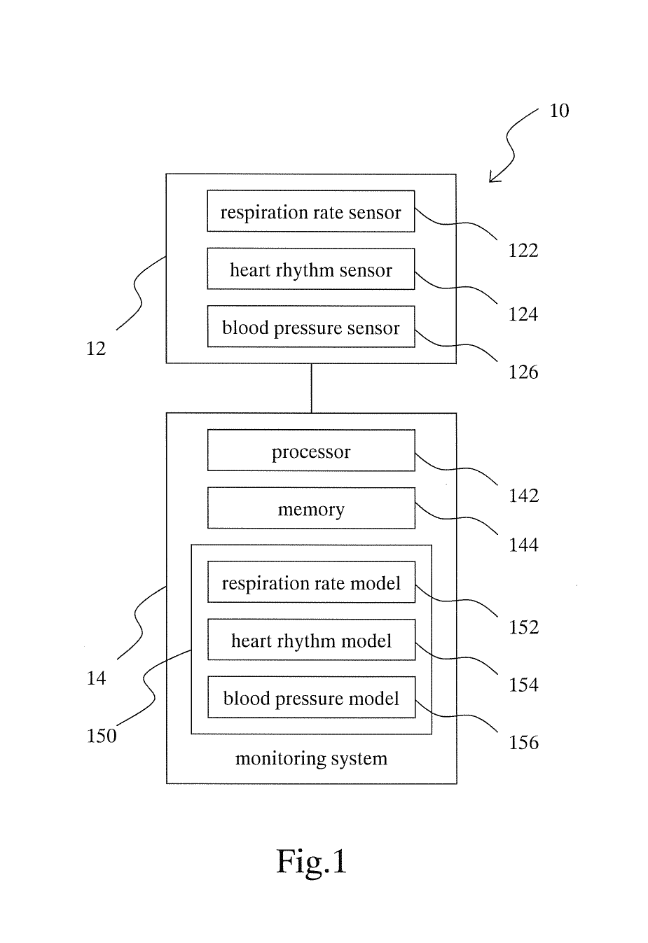

Refer to FIG. 1 a block diagram schematically showing a system for detecting a drive's sudden heart attack according to one embodiment of the present invention. The system 10 for detecting a drive's sudden heart attack of the present invention is built in a microcomputer of a vehicle or an independent device. The system 10 comprises a plurality of sensors 12 and a monitoring system 14. The sensors 12 include a respiration rate sensor 122, a heart rhythm sensor 124 and a blood pressure sensor 126. The heart rhythm sensor 124 is installed in the safety belt or in form of a patch attached to the chest. After the driver wears the safety belt, the heart rhythm sensor 124 touches the chest of the driver and detects the heartbeats of the driver. The respiration rate sensor 122 and the heart rhythm sensor 124 are arranged in an identical patch or respectively in different patches. The respiration rate sensor 122 detects the respiration signals of the driver. In one embodiment, the blood pressure sensor 126 is disposed in an area where the driver's hand holds the steering wheel. The blood pressure sensor detects the blood pressure of the driver in an optical method. For example, the blood pressure sensor 126 projects light to the finger and determines the blood pressure according to the spectrum of the reflected light. Thereby, the three sensors 122, 124 and 126 respectively capture the signals of respiration rate, heart rhythm and blood pressure of the driver. The monitoring system 14 includes a processor 142 and a memory 144. The processor 142 uses the abovementioned signals to establish a plurality of personalized models 150, including a respiration rate model 152, a heart rhythm model 154 and a blood pressure model 156, and stores the models in the memory 144. The processor 142 respectively sets threshold values for the respiration rate model 152, the heart rhythm model 154 and the blood pressure model 156. In addition to establishing the personalized models 150, the processor 142 determines whether any vital-sign signal exceeds the threshold value thereof. If at least one vital-sign signal exceeds the threshold value thereof, the processor 142 determines the risk of the driver according to the number of the types of the vital-sign signals exceeding the threshold values thereof and emits an alert if necessary.

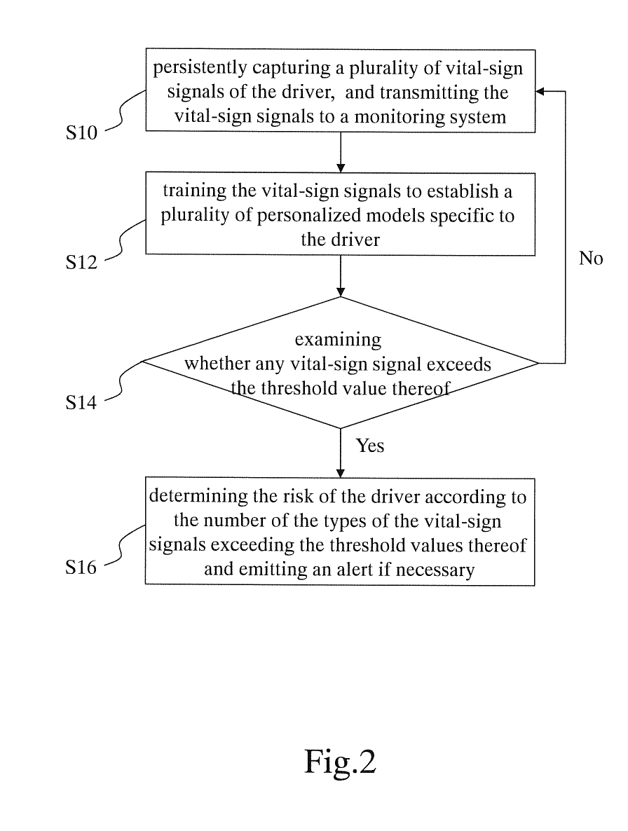

Refer to FIG. 2 a flowchart of a method for detecting a drive's sudden heart attack according to one embodiment of the present invention. In Step S10, at least one sensor respectively captures a plurality of vital-sign signals of the driver, including signals of respiration rate, heart rhythm and blood pressure, and transmits the signals to a monitoring system. The signals are displayed periodically, such as once per 5 minutes. In Step S12, the processor of the monitoring system uses the vital-sign signals to train an artificial neural network to establish a plurality of personalized models that are specific to the driver and respectively have threshold values, including models of respiration rate, heart rhythm, and blood pressure. In Step S14, the monitoring system examines whether any vital-sign signal exceeds the threshold value thereof. If none vital-sign signal exceeds its threshold value, the process returns to Step S10 to continue capturing the vital-sign signals of respiration rate, heart rhythm and blood pressure. If at least one vital-sign signal exceeds the threshold value thereof, the process proceeds to Step S16. In Step S16, the monitoring system determines the risk of the driver according to the number of the types of the vital-sign signals exceeding the threshold values thereof and emits an alert if necessary.

The heart rhythm model is generated via obtaining the electrocardiogram of the driver and using the Fourier Transform to convert the time-domain signals into frequency-domain signals within a range of 0-60 Hz. The artificial neural network is used to train the heartbeats per minute and the frequency-domain signals within a range of 0-60 Hz to generate a personalized heart rhythm model of the driver. The artificial neural network also trains the frequency, intensity and slope of the respiration signals to generate the respiration rate model. The artificial neural network also trains the diastolic blood pressure, the systolic blood pressure, and the mean arterial blood pressure to generate the blood pressure model. The abovementioned three models receives new vital-sign signals to expand the sample groups and continues being trained to approach the status of the driver.

The initial value of the vital-sign threshold can be set to be the commonly expected vital-sign threshold value. For example, the threshold value of the respiration rate model is set be twice the average respiration rate (the number of respirations per minute). The respiration rate below twice the average respiration rate is normal and assigned a value “0”. The respiration rate over twice the average respiration rate is abnormal and assigned a value “1”. The threshold value of the blood pressure model is a systolic pressure of 90 mmHg. The systolic pressure over 90 mmHg is normal and assigned a value “0”. The systolic pressure below 90 mmHg is abnormal and assigned a value “1”. The threshold value of the heart rhythm model is 150 heartbeats per minute. The heart rhythm below 150 heartbeats per minute is normal and assigned a value “0”. The heart rhythm over 150 heartbeats per minute is abnormal and assigned a value “0”. If all the vital-sign signals are normal, the output is (0, 0, 0). If only a vital-sign signal is abnormal, the output is (1, 0, 0), (0, 1, 0), or (0, 0, 1), which indicate a low risk. If two vital-sign signals are abnormal, the output is (1, 1, 0), (0, 1, 1), or (1, 0, 1), which indicate a medium risk. If all the vital-sign signals are abnormal, the output is (1, 1, 1), which indicates a high risk. The above discussion is summarized in Table. 1.

| | Vital Sign | | | | | |
|---|---|---|---|---|---|---|
| | | | | | Blood | |
| | Respiration Rate | | Heart rhythm | | Pressure | |
| Risk | normal | abnormal | normal | abnormal | normal | abnormal |
| None | 0 | — | 0 | — | 0 | — |
| Low | — | 1 | 0 | — | 0 | — |
| | 0 | — | — | 1 | — | — |
| | 0 | — | — | — | — | 1 |
| Medium | — | 1 | — | 1 | 0 | — |
| | 0 | — | — | 1 | — | 1 |
| | — | 1 | 0 | — | — | 1 |
| High | — | 1 | — | 1 | — | 1 |

The initial values of the three threshold values are based on the data of medical periodicals. The artificial neural network trains the personal vital-sign data and modifies the initial values according to the individual condition of a driver. For example, after being trained by the artificial neural network, the threshold values of Driver A are modified to be as follows: a heart rhythm over 130 heartbeats per minute is abnormal; a blood pressure below 80 mmHg is abnormal; a respiration rate over 1.5 times the mean respiration rate is abnormal.

Suppose that the monitoring system detects that one of the three vital-sign signals exceeds its threshold value. For an example, the respiration rate is over twice the mean value. The monitoring system would further examine whether the other two vital-sign signals (heart rhythm and blood pressure) are normal. Suppose that the initial values that have not yet been trained to adapt to an individual are used as the threshold values. If the blood pressure is stable, the driver is regarded as risk-free for the time being. If the blood pressure is unstable, the monitoring system further checks the heart rhythm. If the heart rhythm is below 150 heartbeats per minute, it means that only two of the three vital-sign signals are abnormal. In such a case, the driver should go to the nearby hospital to see a doctor. If the heart rhythm is over 150 heartbeats per minute, it indicates that the driver may highly risk a heart attack. For a further example, the monitoring system detects an abnormal heart rhythm. The monitoring system would further examine the respiration rate signal. If the respiration rate is normal, the driver is regarded as risk-free for the time being. If the respiration rate is abnormal, the monitoring system further examines the blood pressure. If the blood pressure is stable, it means that only two of the three vital-sign signals are abnormal. In such a case, the driver should go to the nearby hospital to see a doctor. If the blood pressure is unstable, it indicates that the driver may highly risk a heart attack. Once the monitoring system confirms that the driver is suffering a heart attack, the monitoring system would immediately trigger the driving control system to brake the vehicle, flash the lights and emit other emergency signals lest the driver keep pushing the accelerator unconsciously and cause a traffic accident.

Some cases may cause the monitoring system to misjudge the status of the driver. For example, talking or laughing may cause abnormal respiration rate; the passenger, cat or dog, which suddenly appears before the vehicle, may cause the heart rhythm to increase and the blood pressure to rise. Therefore, the present invention examines the abovementioned three vital-sign signals simultaneously to exclude misjudgments.

A sudden disease can be verified from the abnormality of any one of blood pressure, heart rhythm and respiration rate. However, a heart attack is normally verified from the abnormality of two or more vital-sign signals. A heart attack must cause abnormal heart rhythm accompanied by dyspnea (abnormal respiration rate) or blood pressure dipping. Therefore, a driver's sudden heart attack must be verified with the three abovementioned vital-sign signals simultaneously.

Different persons have different modes of respiration rate, heart rhythm and blood pressure. Therefore, a person has specific models of respiration rate, heart rhythm and blood pressure, which are established according to the vital-sign signals of the person. The personalized vital-sign models of the present invention will provide valued information while the driver goes to see a doctor.

In conclusion, the present invention proposes a system and method for detecting a driver's sudden heart attack, which captures the signals of heart rhythm, blood pressure and respiration rate simultaneously and uses the signals to train an artificial neural network and establish personalized vital-sign models specific to the driver. The characteristic of simultaneously capturing and separately verifying the three vital-sign signals can increase the accuracy of predicting a sudden heart attack of a driver. Further, the personalized vital-sign models of the present invention provide valued information while the driver goes to see a doctor.

The embodiments have been described in detail to demonstrate the present invention. However, it should be understood: these embodiments are only to exemplify the present invention but not to limit the scope of the present invention. Any equivalent modification or variation according to the characteristic or spirit of the present invention is to be also included within the scope of the present invention.

What is claimed is:

1. A method for detecting a driver's sudden heart attack, applying to detecting whether a driver of a vehicle is suffering a sudden heart attack, and comprising steps:
persistently capturing a plurality of vital-sign signals of said driver and transmitting said vital-sign signals to a monitoring system, wherein said vital-sign signals include a respiration rate signal, a heart rhythm signal and a blood pressure signal;
a processor of said monitoring system using said vital-sign signals to train an artificial neural network and establishing a plurality of personalized models specific to said driver, including a respiration rate model, a heart rhythm model and a blood pressure model, wherein each said personalized model has a threshold value; and
said monitoring system examining whether any one of said vital-sign signals exceeds said threshold value thereof, determining a risk level of said driver according to a quantity of types of said vital-sign signals exceeding said threshold values thereof, and emitting an alert if necessary.

2. The method for detecting a driver's sudden heart attack according to claim 1, wherein said monitoring system is installed in said vehicle and includes a memory recording said vital-sign signals and said personalized models.

3. The method for detecting a driver's sudden heart attack according to claim 1, wherein while only one type of said vital-sign signals exceeds said threshold value thereof, said driver has a low-level risk; while two types of said vital-sign signals exceed said threshold values thereof, said driver has a medium-level risk; while all three types of said vital-sign signals exceed said threshold values thereof, said driver has a high-level risk.

4. The method for detecting a driver's sudden heart attack according to claim 3, wherein said threshold value of said respiration signal is over twice a mean respiration rate.

5. The method for detecting a driver's sudden heart attack according to claim 3, wherein said threshold value of said heart rhythm signal is over 150 heartbeats per minute.

6. The method for detecting a driver's sudden heart attack according to claim 3, wherein said threshold value of said blood pressure signal is a systolic pressure below 90 mmHg.

7. The method for detecting a driver's sudden heart attack according to claim 1, wherein before said personalized models are established, initial values are preset to be said threshold values.

8. A system for detecting a driver's sudden heart attack, applying to detecting whether a driver of a vehicle is suffering a sudden heart attack, and comprising
a plurality of sensors persistently capturing a plurality of vital-sign signals of said driver, wherein said vital-sign signals include a respiration rate signal, a heart rhythm signal and a blood pressure signal; and
a monitoring system including a processor and a memory, wherein said processor uses said vital-sign signals to train a plurality of personalized models, and stores said personalized models in said memory, and wherein said personalized models include a respiration rate model, a heart rhythm model and a blood pressure model, and wherein said processor respectively sets threshold values of said vital-sign signals according to said personalized models, and wherein said processor examines whether any one of said vital-sign signals exceeds said threshold value thereof, determines a risk level of said driver according to a quantity of types of said vital-sign signals exceeding said threshold values thereof, and emits an alert if necessary.

9. The system for detecting a driver's sudden heart attack according to claim 8, wherein while only one type of said vital-sign signals exceeds said threshold value thereof, said driver has a low-level risk; while two types of said vital-sign signals exceed said threshold values thereof, said driver has a medium-level risk; while all three types of said vital-sign signals exceed said threshold values thereof, said driver has a high-level risk.

10. The system for detecting a driver's sudden heart attack according to claim 9, wherein said threshold value of said respiration signal is over twice a mean respiration rate.

11. The system for detecting a driver's sudden heart attack according to claim 9, wherein said threshold value of said heart rhythm signal is over 150 heartbeats per minute.

12. The system for detecting a driver's sudden heart attack according to claim 9, wherein said threshold value of said blood pressure signal is a systolic pressure below 90 mmHg.
